SEQUENCE LISTING (5 sequences)

SEQ ID NO: 1 - protein, 13 aa, Artificial sequence
  Synthetic construct
ELYENKPRRPYIL

SEQ ID NO: 2 - protein, 32 aa, Artificial sequence
  Synthetic construct
FRWGKPVGKKRRPVKVYPNGAEDESAEAFPLE

SEQ ID NO: 3 - protein, 10 aa, Artificial sequence
  Synthetic construct
DRVYIHPFHL

SEQ ID NO: 4 - protein, 17 aa, Artificial sequence
  Synthetic construct
SYSMEHFRWVGKPVGKR

SEQ ID NO: 5 - protein, 22 aa, Artificial sequence
  Synthetic construct
RPVKVYPNGAEDESAEAFPLEF

(19) United States
(12) Patent Application Publication
(10) Pub. No.: US 2005/0250159 A1
(43) Pub. Date: Nov. 10, 2005

(54) Methods for using Raman spectroscopy to obtain a protein profile of a biological sample

(75) Inventors: Xing Su, Cupertino, CA (US); Lei Sun, Santa Clara, CA (US); Mineo Yamakawa, Campbell, CA (US); Tae-Woong Koo, Cupertino, CA (US); Selena Chan, San Jose, CA (US); Andrew A. Berlin, San Jose, CA (US); Narayanan Sundararajan, San Francisco, CA (US)

Correspondence Address: Lisa A. Haile, J.D., Ph.D.;DLA PIPER RUDNICK GRAY CARY US LLP, Suite 1100, 4365 Executive Drive, San Diego, CA, 92121-2133, US

(73) Assignee: INTEL CORPORATION

(21) Appl. No.: 11/083,418

(22) Filed: Mar. 17, 2005

Related U.S. Application Data
(62) Division of application No. 10/749,528, filed on Dec. 30, 2003.

(51) Int. Cl. G01N 33/53; G01N 33/00

(52) U.S. Cl. 435/7.1; 436/86

(57) ABSTRACT

The invention provides methods for analyzing the protein content of a biological sample, for example to obtain a protein profile of a sample provided by a particular individual. The proteins and protein fragments in the sample are separated on the basis of chemical and/or physical properties and maintained in a separated state at discrete locations on a solid substrate or within a stream of flowing liquid. Raman spectra are then detected as produced by the separated proteins or fragments at the discrete locations such that a spectrum from a discrete location provides information about the structure or identity of one or more particular proteins or fragments at the discrete location. The proteins or fragments at discrete locations can be coated with a metal, such as gold or silver, and/or the separated proteins can be contacted with a chemical enhancer to provide SERS spectra. Method and kits for practicing the invention are also provided.

### BACKGROUND OF THE INVENTION

1. Field of the Invention

The present invention relates generally to methods and devices useful to identify the presence of an analyte in a sample and, more particularly, to methods and devices for use of Raman spectroscopy to obtain a protein or peptide profile of a complex biological sample.

2. Background Information

The remarkable success of genome level DNA sequencing has placed us at a threshold of knowledge that was unimaginable 25 years ago. To enable this watershed of data to be transformed into knowledge that will be of use in diagnosing, staging, understanding, and treating human diseases will require that we not only know the sequences of the estimated >30,000 human proteins but also that we identify key changes in protein expression which portend the impending onset of disease. We also need to accurately classify at the molecular level the disease subtype, and understand the functions, interactions, and how to modulate the activities of proteins that are intimately involved in disease processes. One of the most fundamental approaches to understanding protein function is to correlate expression level changes as a function of growth conditions, cell cycle stage, disease state, external stimuli, level of expression of other proteins, or other variable. Although DNA microarray analysis offers a massively parallel approach to genome-wide mRNA expression analysis, there often is not a direct relationship between the in vivo concentration of an mRNA and its encoded protein. Differential rates of translation of mRNAs into protein and differential rates of protein degradation in vivo are two factors that confound the extrapolation of mRNA to protein expression profiles.

Additionally, such microarray analysis is unable to detect, identify or quantify post-translational protein modifications—which often play a key role in modulating protein function. Protein expression analysis offers a potentially large advantage in that it measures the level of the biological effecter protein molecule, not just that of its message. Currently, no protein profiling technology is available that can approach the ability of microarray analysis to simultaneously profile the relative level of mRNA expression of 25,000 or more genes.

Thus, ever increasing attention is being paid to detection and analysis of low concentrations of analytes in various biologic samples. Qualitative analysis of such analytes is generally limited to the higher concentration levels, whereas quantitative analysis usually requires labeling with a radioisotope or fluorescent reagent. Such procedures are generally time consuming and inconvenient. For example, various modes of mass spectroscopy are being widely used for protein profiling (See FIG. 1).

In addition, solid-state sensors and particularly biosensors have received considerable attention lately due to their increasing utility in chemical, biological, and pharmaceutical research as well as disease diagnostics. In general, biosensors consist of two components: a highly specific recognition element and a transducing structure that converts the molecular recognition event into a quantifiable signal. Biosensors have been developed to detect a variety of biomolecular complexes including oligonucleotide pairs, antibody-antigen, hormone-receptor, enzyme-substrate and lectin-glycoprotein interactions. Signal transductions are generally accomplished with electrochemical, field-effect transistor, optical absorption, fluorescence or interferometric devices.

Raman spectroscopy or surface plasmon resonance has also been used seeking to achieve the goal of sensitive and accurate detection or identification of individual molecules from biological samples. When light passes through a medium of interest, a certain amount of the light becomes diverted from its original direction in a phenomenon known as scattering. Some of the scattered light also differs in frequency from the original excitatory light, due to the absorption of light and excitation of electrons to a higher energy state, followed by light emission at a different wavelength. The difference of the energy of the absorbed light and the energy of the emitted light matches the vibrational energy of the medium. This phenomenon is known as Raman scattering, and the method to characterize and analyze the medium or molecule of interest with the Raman scattered light is called Raman spectroscopy. The wavelengths of the Raman emission spectrum are characteristic of the chemical composition and structure of the Raman scattering molecules in a sample, while the intensity of Raman scattered light is dependent on the concentration of molecules in the sample.

A Raman spectrum, similar to an infrared spectrum, consists of a wavelength distribution of bands corresponding to molecular vibrations specific to the sample being analyzed (the analyte). In the practice of Raman spectroscopy, the beam from a light source, generally a laser, is focused upon the sample to thereby generate inelastically scattered radiation, which is optically collected and directed into a wavelength-dispersive spectrometer in which a detector converts the energy of impinging photons to electrical signal intensity.

Historically, the very low conversion of incident radiation to inelastic scattered radiation limited Raman spectroscopy to applications that were difficult to perform by infrared spectroscopy, such as the analysis of aqueous solutions. It was discovered however, that when a molecule in close proximity to a roughened silver electrode is subjected to a Raman excitation source the intensity of the signal generated is increased by as much as six orders of magnitude.

Although the mechanism responsible for this large increase in scattering efficiency is currently the subject of considerable research, it is generally accepted that the phenomenon occurs if the following three conditions are satisfied: (1) that the free-electron absorption of the metal can be excited by light of wavelength between 250 and 2500 nanometers (nm), preferably in the form of laser beams; (2) that the metal employed is of the appropriate size (normally 5 to 1000 nm diameter particles, or a surface of equivalent morphology), and has optical properties necessary for generating a surface plasmon field; and (3) that the analyte molecule has effectively matching optical properties (absorption) for coupling to the plasmon field.

In particular, nanoparticles of gold, silver, copper and certain other metals can function to enhance the localized effects of electromagnetic radiation. Molecules located in the vicinity of such particles exhibit a much greater sensitivity for Raman spectroscopic analysis. SERS is the technique to utilize this surface enhanced Raman scattering effect to characterize and analyze biological molecules of interest.

Sodium chloride and lithium chloride have been identified as chemicals that enhance the SERS signal when applied to a metal nanoparticle or metal coated surface before or after the molecule of interest has been introduced. However, the technique of using these chemical enhancers has not proved sensitive enough to reliably detect low concentrations of analyte molecules, such as single nucleotides or proteins. Only one type of nucleotide, deoxyadenosine monophosphate, and only one type of protein, hemoglobin, have been detected at single molecule level. As a result, SERS has not been viewed as suitable for analyzing the protein content of a complex biological sample, such as blood plasma.

Thus a need exists in the art for a method of analyzing the protein composition of a complex biological sample, such as blood serum that provides more information regarding the characteristics of the proteins and for reliably detecting and/or identifying individual proteins using a Raman spectroscopic analytical technique. In addition, there is also a need in the art for a high throughput means of qualitatively and quantitatively detecting proteins in a complex sample at low concentration levels.

### BRIEF DESCRIPTION OF THE DRAWINGS

FIG. 1 is a block diagram showing the general process used in mass spectrometry for providing a protein profile of a complex biological sample.

FIG. 2 is a block diagram showing the general process used in the invention methods employing SERS spectra for providing a protein profile of a complex biological sample.

FIG. 3 is a drawing showing an apparatus for sample deposition in the invention methods by electro-spray. Protein fragments are separated by HPLC, ionized, and deposited onto a substrate made up of metal islands separated by insulator on a flat surface. Ionized molecules are concentrated on the metal islands after passing a focusing tube. The inner surface of the focusing tube has the same charge as the ionized particles and the collection orifice of the focusing tube is larger than the deposition orifice.

FIG. 4 is a drawing showing another apparatus for sample deposition in the invention methods by wet electro-spray. The separated proteins in hydrophilic solvent are electro-sprayed as ions and adsorbed or covalently linked to modified or unmodified substrate islands separated by a mask or screen on a flat surface for immobilization.

FIG. 5 is compilation of SERS signals obtained from multiple fractions of calf serum peptide chains that have been separated by high pressure liquid chromatography (HPLC).

FIG. 6 is a block diagram showing the invention process for analysis of protein profiling results obtained by Raman spectrometry of a blood sample. The results for an individual sample are integrated into a protein library that is in turn used for further analysis (e.g., diagnosis) of the Raman profile results obtained for an individual test sample.

FIG. 7 is a SERS spectrograph showing SERS spectra collected for standard peptides of Table 1 without the use of Raman tags.

FIG. 8 is a graph showing the results of principal components analysis (PCA) performed on the Raman spectra of FIG. 7

FIGS. 9A and 9B are SERS spectrographs obtained, respectively, for BSA and calf serum without the use of Raman tags.

FIG. 10 is a SERS spectrograph showing SERS spectra obtained from concentrated HPLC-separated fractions of calf serum without the use of Raman labeling.

FIG. 11A is a chromatograph of HPLC-separated trypsin-digested calf serum fractions

FIG. 11B is a graph of UV absorption (215 nm) of the trypsin-digested calf serum fractions whose HPLC chromatograph is shown in FIG. 11A

FIG. 11C is a SERS spectrograph of the trypsin-digested calf serum fractions whose HPLC chromatograph is shown in FIG. 11A.

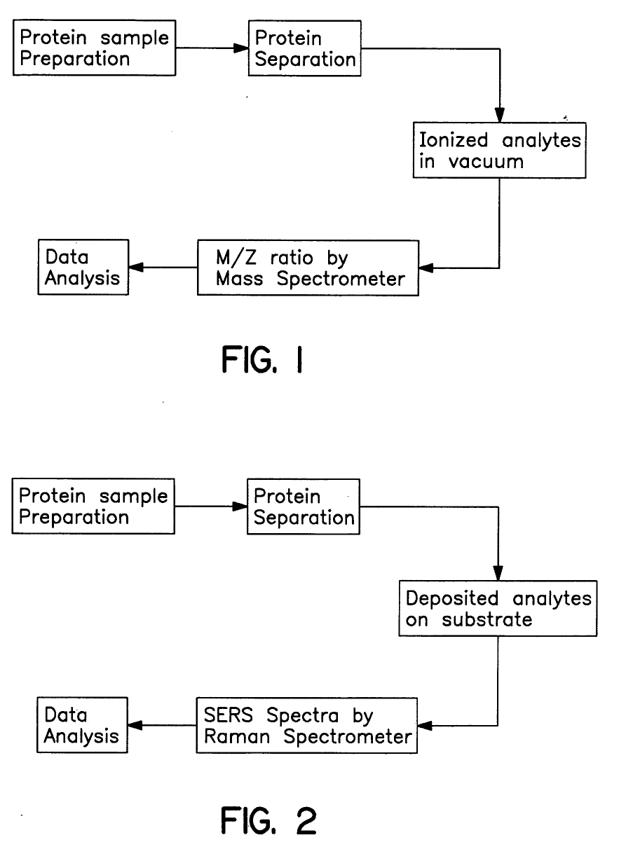

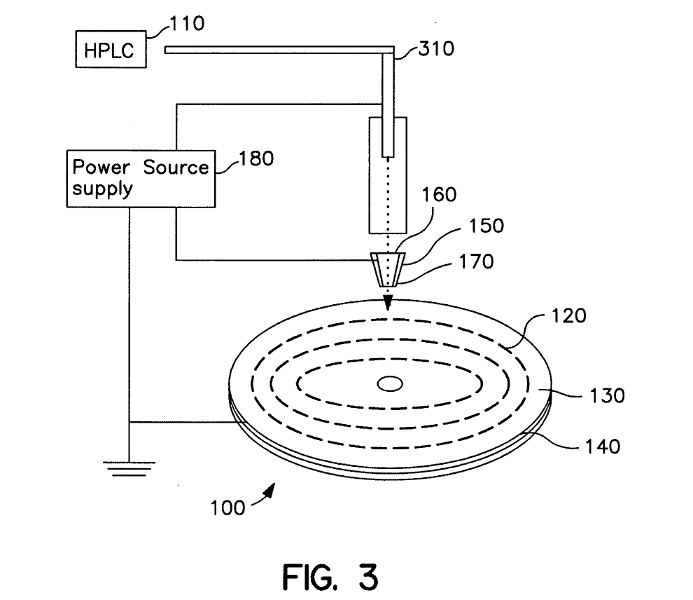

### DETAILED DESCRIPTION OF THE INVENTION

The various embodiments of the invention relate to methods for utilizing Raman spectroscopy to obtain and analyze a protein profile of a complex biological sample. The following detailed description contains numerous specific details in order to provide a more thorough understanding of the disclosed embodiments of the invention. However, it will be apparent to those skilled in the art that the embodiments can be practiced without these specific details. In other instances, devices, methods, procedures, and individual components that are well known in the art have not been described in detail herein.

The invention provides methods for analyzing the protein content of a biological sample by separating the proteins and protein fragments in the sample on the basis of chemical and/or physical properties of the proteins and maintaining the separated proteins in a separated state at discrete locations on a solid substrate or within a stream of flowing liquid. Raman spectra are then detected as produced by the separated proteins at the discrete locations, wherein the spectrum from a discrete location provides information about the structure of one or more particular proteins at the discrete location.

In another embodiment, the invention provides kits for analyzing the protein composition of a complex mixture of proteins, such kits including a substrate having a plurality of discrete locations that are coated with positively charged or negatively charged compounds, or with neutral or hydrophobic polymers for immobilization of proteins and protein fragments at the discrete locations; and a container holding ions of silver, gold, copper or aluminum.

In yet another embodiment, the invention provides systems for analyzing the protein composition of a complex mixture of proteins. The invention systems comprising such components as a substrate with a plurality of discrete locations having a coating selected from a metal layer, a positively charged or negatively charged compound, or neutral or hydrophobic polymer for immobilization of proteins and protein fragments. The invention systems further comprise a sample containing at least one protein-containing compound, a Raman spectrometer, and a computer comprising an algorithm for analysis of the sample.

The following paragraphs discuss a variety of concepts and terms that will be useful in understanding the various embodiments of the invention.

The term “complex biological sample” as used herein means a sample containing hundreds of protein-containing analytes, such as a body fluid from a host. The sample can be examined directly or can be pretreated to denature or fragment the protein-containing molecules in the sample to render them more readily detectible. Furthermore, the analyte of interest can be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The body fluid can be, for example, urine, blood, plasma, serum, saliva, semen, stool, sputum, cerebral spinal fluid, tears, mucus, and the like.

As used herein, the term “protein” encompasses peptides, polypeptides, and proteins as well as such protein-containing analytes as antigens, glycoproteins, lipoproteins, and the like.

In one embodiment according to the invention, methods are provided for obtaining protein composition information from a complex biological mixture, such as a patient sample. Proteins in the biological sample are solubilized in an aqueous solution or hydrophilic solvent. Optionally, the proteins in the sample can be denatured using agents selected from reducing agents, surfactants, chaotropic salts, and the like. Common chemicals that can be used to reduce disulfide bonds include without limitation DTT, DTE, 2-mercaptoethanol, and the like. Representative surfactants that can be used to denature proteins include without limitation sodium dodecyl sulfate (SDS), lithium dodecyl sulfate (LDS), Triton X 100®, Tween-20®, and the like. Typical chaotropic salts that can be used to denature proteins include without limitation GuSCN, NaSCN, GuClO₄, Na ClO₄, and urea. Protein fragmentation is another way of denaturing proteins, and can be accomplished using a chemical cleavage agent or a serine-protease, such as trypsin, for digestion of the proteins. Proteins can also be maintained in native structures (non-denatured) for Raman spectroscopic or SERS analysis.

To increase accuracy and resolution, proteins or protein fragments in the sample under analysis are separated according to their chemical and physical properties using any of a number of known methods. Size separation is based, for example, on physical size or molecular weights (mass). Charge separation is based on surface charges, or iso-electric points. Hydrophilicity separation is based on interaction of the proteins or fragments with hydrophobic medium. Affinity separation is based on sequence structure and conformation. A common mode of protein separation is liquid chromatography. Non-limiting methods of protein and peptide separation include size exclusion, reverse phase, ion exchange, affinity (using FPLC, regular chromatography or microfluidic devices), and electrophoresis (such as capillary electrophoresis or chip electrophoresis). Any of these techniques, as well as others known in the art, can be used alone or in combination for protein separation.

Once separated, proteins or fragments in the sample are maintained in a separated state. In one embodiment of the invention methods, the separated proteins or fragments are maintained in a separated state by deposition and immobilization in discrete spaced locations on a solid surface. Methods for sample deposition and immobilization include contact writing, contact spotting, liquid spraying, dry particle spraying (i.e. electro-spray), and the like. In electrospray applications, the proteins or protein fragments are subjected to an electric field to cause ionization of the proteins or particles to aid in guiding the analytes to particular discrete locations of substrate.

Nano-electrospray technology is widely used in mass spectrometry, and nano-electrospray ion sources are known in the art. These miniaturized electrospray sources consist of a metallized glass capillary needle with a tip of inner diameter of about 1 μm from which the analyte solution is sprayed. Droplets produced by the nano-electrospray are about 100 times smaller in volume than those in conventional electrospray sources, allowing efficient use of the sample without loss of material in large droplets, from which peptides cannot be ionized. The ion current is increased even though the flow rate through the capillary needle is extremely low (20-40 nl min⁻¹). Since very small amounts (1-2 μl) of the protein containing mixture can be subjected to nano-electrospray mass spectrometry, it is contemplated that the feed stream to the nano-spray deposition device can be obtained from a nano-electrospray mass spectrometer, which is used to separate the proteins in the sample. Techniques of electrospray and nano-electrospray and their uses are summarized in Covey, T. R. and Devan, P. Nanospray Electrospray Ionization Development: LC/MS, CE/MS Application. Practical Spectroscopy Series, Volume 32: Applied Electrospray Mass Spectrometry; Pramanik, B. N.; Ganguly, A. K.; Gross, M. L., Eds.; Marcel Dekker: New York, N.Y., 2002.

The size of the substrate array will depend on the end use of the array. Arrays containing from about 10 to many millions of different discrete substrate sites can be made. Generally, the array will comprise from 10 or more to as many as a billion or more such sites, depending on the size of the surface. Thus, very high density, high density, moderate density, low density or very low density arrays can be made. Some ranges for very high-density arrays are from about 10,000,000 to about 2,000,000,000 sites per array. High-density arrays range from about 100,000 to about 10,000,000 sites. Moderate density arrays range from about 10,000 to about 50,000 sites. Low-density arrays are generally less than 10,000 sites. Very low-density arrays are less than 1,000 sites.

The sites can comprise a pattern, i.e. a regular design or configuration, or can be randomly distributed. For example, a regular pattern of sites can be used such that the sites can be addressed in an X-Y coordinate plane. The surface of the substrate can be modified to allow attachment of analytes at individual sites. Thus, the surface of the substrate can be modified such that discrete sites are formed. In one embodiment, the surface of the substrate can be modified to contain wells, i.e. depressions in the surface of the substrate. This can be done using a variety of known techniques, including, but not limited to, photolithography, stamping techniques, molding techniques and microetching techniques. As will be appreciated by those in the art, the technique used will depend on the composition and shape of the substrate. Alternatively, the surface of the substrate can be modified to contain chemically derived sites that can be used to attach analytes or probes to discrete locations on the substrate. The addition of a pattern of chemical functional groups, such as amino groups, carboxy groups, oxo groups and thiol groups can be used to covalently attach molecules containing corresponding reactive functional groups or linker molecules.

The size of the discrete locations is generally in the range from about 0.1 μm to 10 mm, for example 1 μm to 1 mm, or 5 μm to 500 μm.

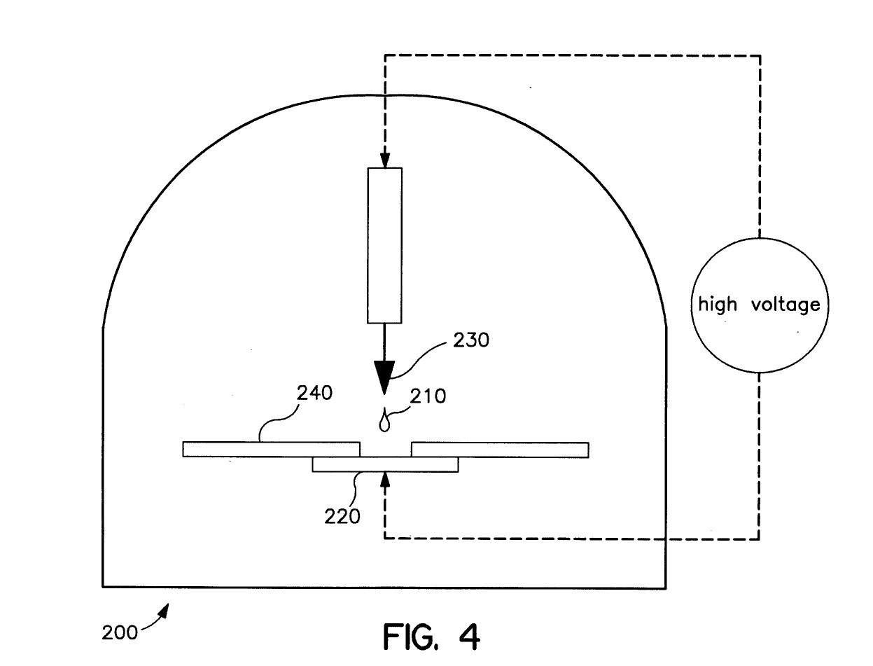

FIG. 3 shows an example of sample deposition by wet electro-spray 100. Protein fragments (dotted line) having been separated by HPLC 110, are ionized by an electric field provided by power source 180, and deposited onto a substrate made up of metal islands 120 separated by insulator 130 on a flat surface 140. Ionized molecules are concentrated on the metal islands after passing a focusing tube 150. The inner surface of the focusing tube has the same charge as the ionized particles and the collection orifice 160 of the focusing tube is larger than the deposition orifice 170. Water molecules are removed in the electro-spray process, as is known in the art. Alternatively, the samples can be deposited and immobilized without denaturing using wet electrospray deposition on a substrate such as aluminum as described in Anal. Chem., 2001, 73:6047. In this wet electrospray technique functionally active protein films are fabricated which retain native properties of the proteins.

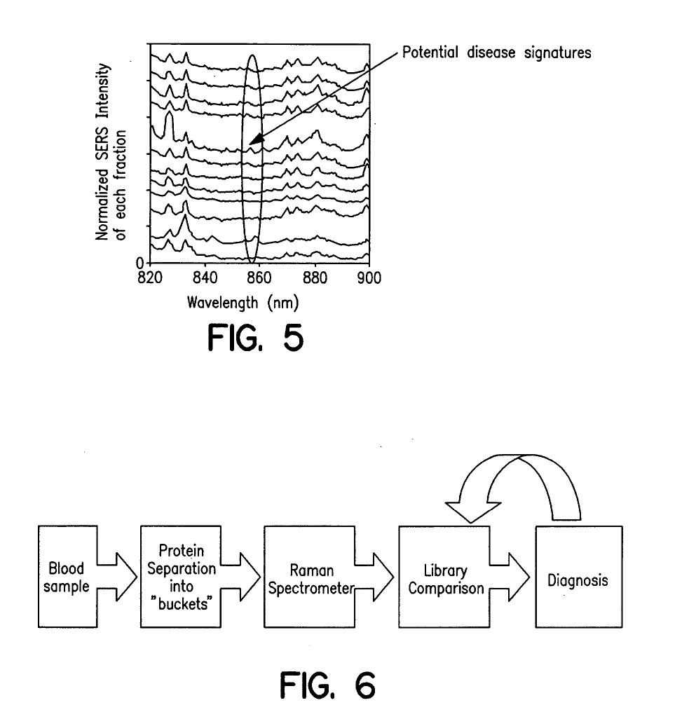

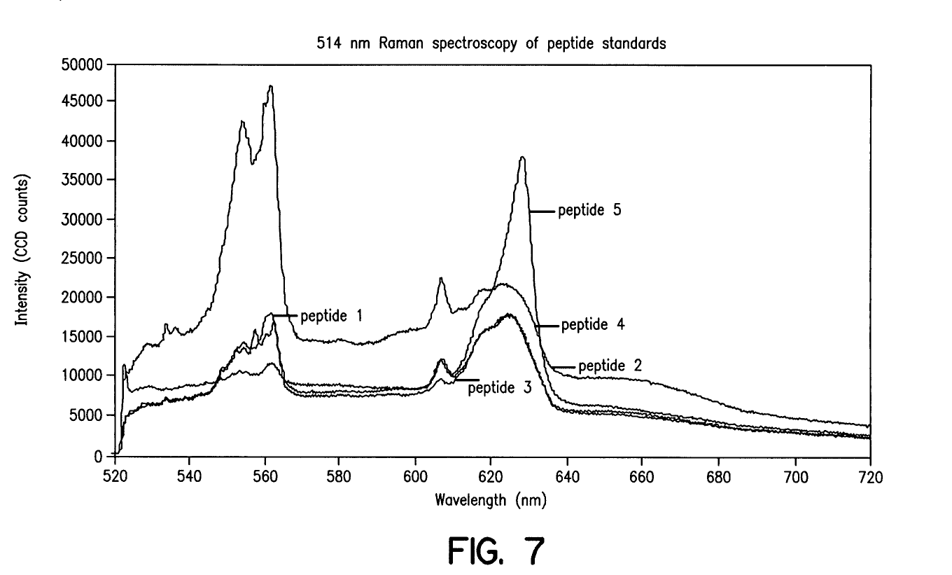

In the electro-spray device 200, as shown in FIG. 4, a protein in hydrophilic solvent 210 is electro-sprayed from a capillary 230 and adsorbed or covalently linked to modified or unmodified substrate island 220 formed by mask 240 for immobilization. When fixed in a separated state using this technique, functionally active proteins tend to retain more specific/unique molecular signatures for scanning analysis, such as Raman scanning, due to their intact three-dimensional conformations and spatial relationships among intermolecular chemical bonds. Mechanisms (substrates or devices) that concentrate proteins or peptides before or subsequent to immobilization can also be used.

Materials for direct analyte contact are referred to herein as substrates and comprise metal, such as gold, silver, copper, and aluminum, or materials, such as silicon, glass and ceramics. The substrate can also be a flat and/or porous surface, and in order to increase the interaction of the proteins with the solid substrate, the substrate can be coated with positively charged or negatively charged compounds, or with neutral or hydrophobic polymers. After deposition, the separated analytes can be heated or baked on the substrate, sufficient to further immobilize the analytes on the surface, for example at 100° C. for about 2 hours.

For preparation of SERS-active nanoparticles, the immobilized proteins and/or fragments immobilized on the substrate are contacted with silver colloid particles, as is known in the art and as described herein, in individual or aggregate forms in the presence of chemical enhancer salts, such as LiCl or NaCl. A Raman spectrometer is used to collect the spectroscopic signals from sample areas. For samples with high concentration of proteins, ordinary Raman spectroscopy can be used to accurately quantify the amount of proteins. The relation between spectra and sample positions are recorded and correlated.

Alternatively, proteins from a sample can also be maintained in a separated state in a stream of liquid within a microfluidic system. Optionally, the separated protein samples in liquid can be mixed with a stream of metal colloids in another fluid stream to allow SERS detection of protein fragments without immobilizing the analytes. A number of mixing strategies in microfluidic environments are available in the literature (Stroock et al., Science 295, 647 (2002); Johnson et. al., Anal. Chem., (2001)). Alternatively, a stream of metal colloids could be mixed with the protein fragments sample outlet stream using a simple micromixing tee from Upchurch Scientific Inc. for subsequent SERS detection.

Raman spectra and/or SERS scanning is performed to analyze the protein and/or protein fragments immobilized at discrete locations on the substrate or in the flowing liquid using techniques known in the art and as described herein to obtain information regarding the protein composition of the sample under analysis.

Data Analysis.

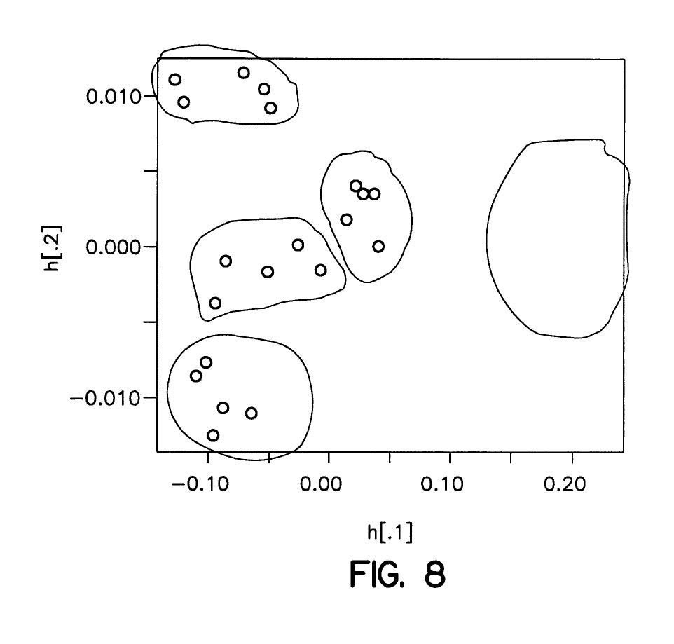

The spectral profiles (or molecular signatures) collected for a sample are analyzed based on a conventional spectrum analysis, which typically involves peak and baseline analysis, system noise, quantization, protocol error analysis, and the like. Peak location, line shapes and relative peak intensities in a spectrum from a given substrate location are analyzed and the relative concentrations of different proteins are estimated from this information. Statistical multivariate analysis techniques, such as principal components analysis (PCA) can also be used for analyzing the Raman spectra. For example, the intensities of the spectral features from protein backbones, such as amide groups, can be used to quantify the total amount of the proteins at a particular substrate location. Proteins with various chemical bands will show different identifiable spectral features, and the presence of certain proteins can be detected by their spectral features. In addition to the protein profile obtained by these methods for the sample, certain additional sample information (e.g., patient information, control identification, or experimental conditions) can be correlated to the profiling information. For example, spectrum information can be correlated with such information as whether the sample comes from a patient with a particular disease or taking a particular drug with a particular desired outcome. Such information can be compiled from a large number of parallel samples (large enough to be statistically significant) so as to form a protein library for a particular type of sample, such as blood serum, to create a database of medically relevant Raman signature information. In addition, the profile from an individual sample can be compared with an existing protein library to determine anomalies or differences between the norm of profiles in the library and a patient sample. FIG. 6 is a block diagram showing this process. Such techniques are useful for such purposes as drug development, clinical diagnosis or biomedical research.

In another embodiment the invention provides kits for analyzing the protein composition of a complex mixture of proteins, such kits comprising a substrate having a plurality of discrete locations that are coated with positively charged or negatively charged compounds, or with neutral or hydrophobic polymers for immobilization of proteins and protein fragments at the discrete locations; and a container holding ions of silver, gold, copper or aluminum. The invention kit can optionally further comprise a protein denaturing agent.

In still another embodiment of the invention, there are provided systems for analyzing the protein composition of a complex mixture of proteins comprising such components as a substrate with a plurality of discrete locations having a coating selected from a metal layer, a positively charged or negatively charged compound, or neutral or hydrophobic polymer for immobilization of proteins and protein fragments. The invention systems further comprise a sample containing at least one protein-containing compound, a Raman spectrometer; and a computer comprising an algorithm for analysis of the sample. In one embodiment, the Raman spectrometer is a scanner of SERS signals received consecutively from a plurality of the discrete locations, such as is useful for high throughput screening of the sample contents immobilized at the discrete locations. In one aspect of the invention methods, the SERS-active nanoparticles incorporated into the invention gel matrix and used in certain other analyte separation techniques described herein are composite organic-inorganic nanoparticle (“COINs”). These SERS-active probe constructs comprise a core and a surface, wherein the core comprises a metallic colloid comprising a first metal and a Raman-active organic compound. The COINs can further comprise a second metal different from the first metal, wherein the second metal forms a layer overlying the surface of the nanoparticle. The COINS can further comprise an organic layer overlying the metal layer, which organic layer comprises the probe. Suitable probes for attachment to the surface of the SERS-active nanoparticles include, without limitation, antibodies, antigens, polynucleotides, oligonucleotides, receptors, ligands, and the like.

The metal required for achieving a suitable SERS signal is inherent in the COIN, and a wide variety of Raman-active organic compounds can be incorporated into the particle. Indeed, a large number of unique Raman signatures can be created by employing nanoparticles containing Raman-active organic compounds of different structures, mixtures, and ratios. Thus, the methods described herein employing COINs are useful for the simultaneous detection of many analytes in a sample, resulting in rapid qualitative analysis of the contents of “profile” of a body fluid. In addition, since many COINs can be incorporated into a single nanoparticle, the SERS signal from a single COIN particle is strong relative to SERS signals obtained from Raman-active materials that do not contain the nanoparticles described herein. This situation results in increased sensitivity compared to Raman-techniques that do not utilize COINs.

As used herein, the term “colloid” refers to nanometer size metal particles suspending in a liquid, usually water. Typical metals contemplated for use in invention nanoparticles include the coinage metals, for example, silver, gold, platinum, aluminum, and the like.

As used herein, “Raman-active organic compound” refers to an organic molecule that produces a unique SERS signature in response to excitation by a laser. A variety of Raman-active organic compounds are contemplated for use as components in COINs. In certain embodiments, Raman-active organic compounds are polycyclic aromatic or heteroaromatic compounds. Typically the Raman-active compound has a molecular weight less than about 300 Daltons.

Additional, non-limiting examples of Raman-active organic compounds include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, carboxyfluorescein, 5-carboxy rhodamine, 6-carboxyrhodamine, 6-carboxytetramethyl amino phthalocyanines, azomethines, cyanines, xanthines, succinylfluoresceins, aminoacridine, and the like. These and other Raman-active organic compounds can be obtained from commercial sources (e.g., Molecular Probes, Eugene, Oreg.).

In certain embodiments, the Raman-active compound is adenine, adenine, 4-amino-pyrazolo(3,4-d)pyrimidine, 2-fluoroadenine, N6-benzolyadenine, kinetin, dimethyl-allyl-amino-adenine, zeatin, bromo-adenine, 8-aza-adenine, 8-azaguanine, 6-mercaptopurine, 4-amino-6-mercaptopyrazolo(3,4-d)pyrimidine, 8-mercaptoadenine, or 9-amino-acridine, 4-amino-pyrazolo(3,4-d)pyrimidine, or 2-fluoroadenine. In one embodiment, the Raman-active compound is adenine.

When fluorescent compounds are incorporated into COINs and other Raman-active probe constructs described herein, the fluorescent compounds can include, but are not limited to, dyes, intrinsically fluorescent proteins, lanthanide phosphors, and the like. Dyes useful for incorporation into COINs and other Raman-active probe constructs or constructs providing an optical signal include, for example, rhodamine and derivatives, such as Texas Red, ROX (6-carboxy-X-rhodamine), rhodamine-NHS, and TAMRA (5/6-carboxytetramethyl rhodamine NHS); fluorescein and derivatives, such as 5-bromomethyl fluorescein and FAM (5′-carboxyfluorescein NHS), Lucifer Yellow, IAEDANS, 7-Me₂, N-coumarin-4-acetate, 7-OH-4-CH₃ -coumarin-3-acetate, 7-NH₂ -4CH₃ -coumarin-3-acetate (AMCA), monobromobimane, pyrene trisulfonates, such as Cascade Blue, and monobromotrimethyl-ammoniobimane.

COINs are readily prepared for use in the invention methods using standard metal colloid chemistry. The preparation of COINs also takes advantage of the ability of metals to adsorb organic compounds. Indeed, since Raman-active organic compounds are adsorbed onto the metal during formation of the metallic colloids, many Raman-active organic compounds can be incorporated into the COIN without requiring special attachment chemistry.

In general, the COINs used in the invention methods are prepared as follows. An aqueous solution is prepared containing suitable metal cations, a reducing agent, and at least one suitable Raman-active organic compound. The components of the solution are then subject to conditions that reduce the metallic cations to form neutral, colloidal metal particles. Since the formation of the metallic colloids occurs in the presence of a suitable Raman-active organic compound, the Raman-active organic compound is readily adsorbed onto the metal during colloid formation. This simple type of COIN is referred to as type I COIN. Type I COINs can typically be isolated by membrane filtration. In addition, COINs of different sizes can be enriched by centrifugation.

In alternative embodiments, the COINs can include a second metal different from the first metal, wherein the second metal forms a layer overlying the surface of the nanoparticle. To prepare this type of SERS-active nanoparticle, type I COINs are placed in an aqueous solution containing suitable second metal cations and a reducing agent. The components of the solution are then subject to conditions that reduce the second metallic cations so as to form a metallic layer overlying the surface of the nanoparticle. In certain embodiments, the second metal layer includes metals, such as, for example, silver, gold, platinum, aluminum, and the like. This type of COIN is referred to as type II COINs. Type II COINs can be isolated and or enriched in the same manner as type I COINs. Typically, type I and type II COINs are substantially spherical and range in size from about 20 nm to 60 nm. The size of the nanoparticle is selected to be very small with respect to the wavelength of light used to irradiate the COINs during detection.

Typically, organic compounds are attached to a layer of a second metal in type II COINs by covalently attaching organic compounds to the surface of the metal layer Covalent attachment of an organic layer to the metallic layer can be achieved in a variety ways well known to those skilled in the art, such as for example, through thiol-metal bonds. In alternative embodiments, the organic molecules attached to the metal layer can be crosslinked to form a molecular network.

The COIN(s) used in the invention methods can include cores containing magnetic materials, such as, for example, iron oxides, and the like. Magnetic COINs can be handled without centrifugation using commonly available magnetic particle handling systems. Indeed, magnetism can be used as a mechanism for separating biological targets attached to magnetic COIN particles tagged with particular biological probes.

In the invention systems and in practice of the invention methods, the Raman spectrometer can be part of a detection unit designed to detect and quantify Raman signals obtained by the invention methods by Raman spectroscopy. Methods for detection of Raman signals, for example from proteins associated with metal nanoparticles, using Raman spectroscopy are known in the art. (See, e.g., U.S. Pat. Nos. 5,306,403; 6,002,471; 6,174,677). Variations on surface enhanced Raman spectroscopy (SERS), surface enhanced resonance Raman spectroscopy (SERS) and coherent anti-Stokes Raman spectroscopy (CARS) have been disclosed.

A non-limiting example of a Raman detection unit is disclosed in U.S. Pat. No. 6,002,471. An excitation beam is generated by either a frequency doubled Nd:YAG laser at 532 nm wavelength or a frequency doubled Ti:sapphire laser at 365 nm wavelength. Pulsed laser beams or continuous laser beams can be used. The excitation beam passes through confocal optics and a microscope objective, and is focused onto the flow path and/or the flow-through cell. The Raman emission light from the separated proteins is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector, that includes an avalanche photodiode or CCD array interfaced with a computer for counting and digitization of the signal.

Another example of a Raman detection unit is disclosed in U.S. Pat. No. 5,306,403, including a Spex Model 1403 double-grating spectrophotometer with a gallium-arsenide photomultiplier tube (RCA Model C31034 or Burle Industries Model C3103402) operated in the single-photon counting mode. The excitation source includes a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 nm (U.S. Pat. No. 6,174,677), a light emitting diode, an Nd:YLF laser, and/or various ions lasers and/or dye lasers. The excitation beam can be spectrally purified with a bandpass filter (Corion) and can be focused on the flow path of discrete locations in a flowing carrier stream or discrete locations on a solid substrate using a 6× objective lens (Newport, Model L6×). The objective lens can be used to both excite the Raman-active proteins associated with the metal nanoparticles and to collect the Raman signal, by using a holographic beam splitter (Kaiser Optical Systems, Inc., Model HB 647-26N18) to produce a right-angle geometry for the excitation beam and the emitted Raman signals. A holographic notch filter (Kaiser Optical Systems, Inc.) can be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors can be used, such as Fourier-transform spectrographs (based on Michaelson interferometers), charged injection devices, photodiode arrays, InGaAs detectors, electron-multiplied CCD, intensified CCD and/or phototransistor arrays.

Any suitable form or configuration of Raman spectroscopy or related techniques known in the art can be used for detection of Raman signals (such as SERS signals) from the proteins in practice of the invention methods, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

In certain aspects of the invention, a system for detecting the Raman signals produced by particular proteins in practice of the invention methods includes an information processing system. An exemplary information processing system may incorporate a computer that includes a bus for communicating information and a processor for processing information. In one embodiment of the invention, the processor is selected from the Pentium® family of processors, including without limitation the Pentium® II family, the Pentium® III family and the Pentium® 4 family of processors available from Intel Corp. (Santa Clara, Calif.). In alternative embodiments of the invention, the processor can be a Celeron®, an Itanium®, or a Pentium Xeon® processor (Intel Corp., Santa Clara, Calif.). In various other embodiments of the invention, the processor can be based on Intel® architecture, such as Intel® IA-32 or Intel® IA-64 architecture. Alternatively, other processors can be used. The information processing and control system may further comprise any peripheral devices known in the art, such as memory, display, keyboard and/or other devices.

In particular examples, the detection unit can be operably coupled to the information processing system. Data from the detection unit can be processed by the processor and data stored in memory. Data on emission profiles for various patient samples may also be stored in memory. The processor may compare the emission spectra from a discrete “spot” on the substrate to a compilation of data obtained from analysis of a plurality of similar patient samples to identify a particular protein or fragment in the sample or to identify a difference between the protein in the sample under analysis and corresponding proteins in the protein library. The processor may analyze the data from the detection unit to determine, for example, the presence of a post-translational modification in a particular protein that is not present in corresponding proteins of healthy individuals. The information processing system may also perform standard procedures such as subtraction of background signals.

While certain methods of the present invention can be performed under the control of a programmed processor, in alternative embodiments of the invention, the methods can be fully or partially implemented by any programmable or hardcoded logic, such as Field Programmable Gate Arrays (FPGAs), TTL logic, or Application Specific Integrated Circuits (ASICs). Additionally, the disclosed methods can be performed by any combination of programmed general purpose computer components and/or custom hardware components.

Following the data gathering operation, the data will typically be reported to a data analysis operation. To facilitate the analysis operation, the data obtained by the detection unit will typically be analyzed using a digital computer such as that described above. Typically, the computer will be appropriately programmed for receipt and storage of the data from the detection unit as well as for analysis and reporting of the data gathered.

In certain embodiments of the invention, custom designed software packages can be used to analyze the data obtained from the detection unit. In alternative embodiments of the invention, data analysis can be performed, using an information processing system and publicly available software packages.

The invention is further illustrated by the following non-limiting examples.

### EXAMPLE 1

Experiments on Standard Peptides.

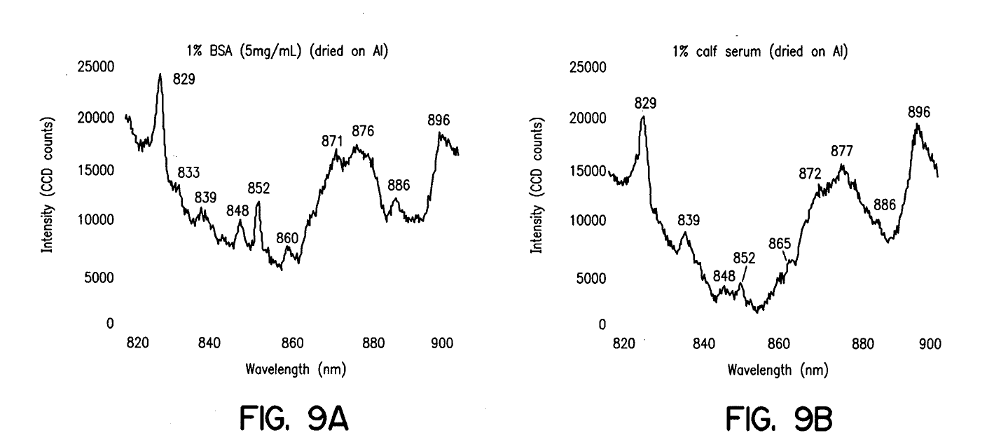

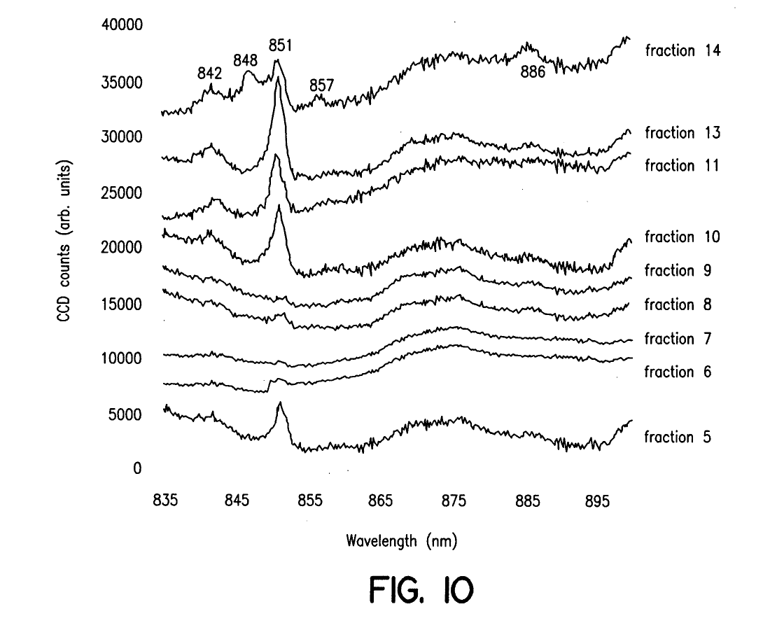

Standard peptides as shown in Table 1 below were synthesized and 10 μl of stock solution (100 ng/μl) of standard peptides was deposited onto discrete locations on an aluminum substrate and left to dry. Raman spectroscopy was performed with SERS colloidal solution of 80 μl of 1:2 colloidal silver/water and 20 nl of 0.5 M LiCI. A total of 1500 spectra were collected for each peptide: 5 experiments @ 3 scans each for 100 frames. To understand whether Raman signals of peptides can be distinguished after normalization of spectra, principal components analysis (PCA) was performed. FIG. 7 shows the Raman spectra collected for each of the peptides. The results of the PCA analysis are shown in FIG. 8.

TABLE 1
Peptide Standards

| | |
|---|---|
| sample ID | Description |

| | |
|---|---|
| 1 | Neurotensin - pGlu-Leu-Tyr-Glu-Asn-Lys- |
| | Pro-Arg-Arg-Pro-Tyr-Ile-Leu |
| | (SEQ ID NO: 1) |
| 2 | ACTH (7-38) - Phe-Arg-Trp-Gly-Lys-Pro-Val- |
| | Gly-Lys-Lys-Arg-Arg-Pro-Val-Lys-Val-Tyr- |
| | Pro-Asn-Gly-Ala-Glu-Asp-Glu-Ser-Ala-Glu- |
| | Ala-Phe-Pro-Leu-Glu (SEQ ID NO: 2) |
| 3 | Angiotensin I - Asp-Arg-Val-Tyr-Ile-His- |
| | Pro-Phe-His-Leu (SEQ ID NO: 3) |
| 4 | ACTH (1-17) - Ser-Tyr-Ser-Met-Glu-His- |
| | Phe-Arg-Trp-Val-Gly-Lys-Pro-Val-Gly-Lys- |
| | Arg (SEQ ID NO: 4) |
| 5 | ACTH (18-39) - Arg-Pro-Val-Lys-Val-Tyr- |
| | Pro-Asn-Gly-Ala-Glu-Asp-Glu-Ser-Ala-Glu- |
| | Ala-Phe-Pro-Leu-Glu-Phe (SEQ ID NO: 5) |

### EXAMPLE 2

The purpose of this experiment was to determine optimal sample detection conditions for obtaining a protein profile of a model complex protein without Raman tagging or labeling of protein targets. Reagent grade calf cell culture serum was used as the sample source. In the first experiment, three sets of samples of whole calf serum deposited on aluminum substrate and either air-dried or wet tested after each step in application of a covering of colloidal silver (containing 160 μL Ag in 1:2 dilution with water)+BSA (20 μL 1% BSA)+LiCl (40 μL 0.5M LiCl) were prepared. Table 2 below shows the combinations of sample detection conditions for each sample. The samples were excited at a wavelength range from 820 to 900 and the SERS signals were collection for 1 sec. tests. Only samples 5 (wet-dry-wet) and 9 (wet-wet-wet) yielded SERS spectra, showing that wet samples are preferable to dry samples under these conditions.

TABLE 2

| | Colloidal Silver | | |
|---|---|---|---|
| Sample # | Soln. | BSA | LiCl |
| 1 | Wet | Dry | Dry |
| 2 | Dry | Wet | Dry |
| 3 | Dry | Dry | Wet |
| 4 | Wet | Wet | Dry |
| 5 | Wet | Dry | Wet |
| 6 | Dry | Wet | Wet |
| 7 | Dry | Dry | Dry |
| 8 | Wet | Wet | Wet |

To determine the SERS detection limit for whole calf serum, the experiment above was repeated using progressively more dilute preparations of calf serum in water. By this means it was determined that the SERS detection limit for whole calf serum is 0.1% calf serum in water (using 785 nm excitation wavelength, 1 sec collection time).

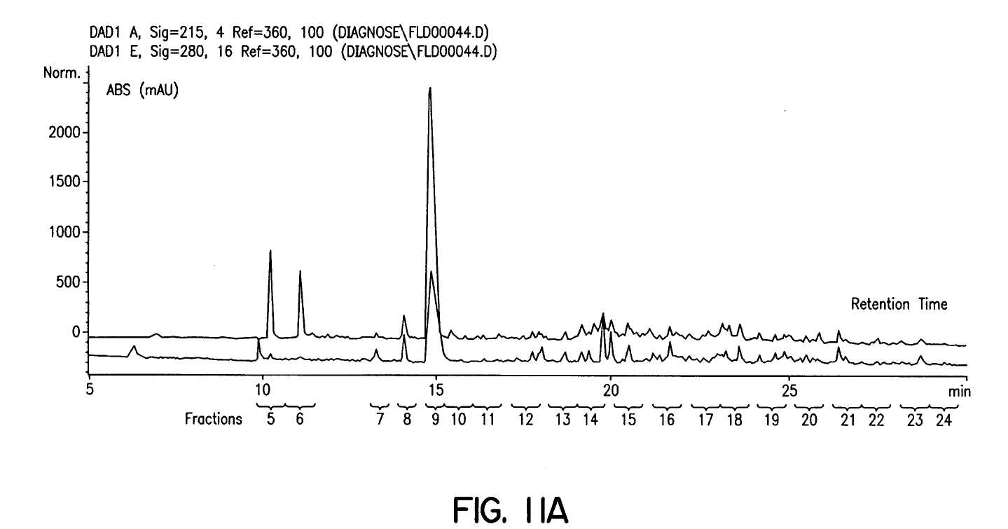

SERS spectra of 1% BSA and 1% calf serum (air dried on aluminum substrate) were collected (1 sec collection time) and compared. As shown in FIGS. 9A and 9B, BSA and calf serum, respectively, have similar SERS spectra. To understand the similarities as well as the differences, SERS spectra were collected using BSA from two different vendors: New England Biolabs (3.33 mg/ml BSA in 1× PBS) and from Roche Chemicals (2.5 mg/ml BSA in 1× PBS). The New England Biolabs BSA sample yielded a much stronger SERS signals than the Roche Chemicals sample (using spectral collection from 820 to 900 nm for 1 sec). It was hypothesized that possible differences of purity or acetylated modification in BSA among vendors would account for the differences in SERS spectra.

### EXAMPLE 3

The purpose of this experiment was to determine optimal conditions for obtain SERS data from HPLC separated protein fractions of a complex protein sample containing intact proteins, using calf serum as the model sample. In preparation for this experiment, low molecular weight protein standards were fractionated by HPLC. A concentration of 1.33 μg/μl in 1× PBS was used for each, with an injection volume of 10 μl. The standards used were Phosphorylase (97 kDa); BSA (66 kDa); Ovalbumin (45 kDa); Carbonic anhydrase (30 kDa); Trypsin inhibitor (20.1 kDa); and Alpha-lactalbumin (14.4 kDa).

After filtering with a 0.45 μm spin filter by centrifuging at 14000 rpm for 10 minutes, calf serum was separated by HPLC at 1:30 dilution in water using a Zorbax GF-220 column and injection volume of 10 μl. Fractions 1-11 were collected over 12.5 min elution time and one additional fraction was collected after about 20 min. It was found that the concentrations of proteins were very low to obtain UV-Vis measurements. Therefore, a protein assay calibration kit (Micro BCA) was used to stain the proteins purple and fractions were read at A₅₆₂. However concentrations were still low, except for fraction 10, which was probably BSA. An albumin depletion kit was unable to sufficiently reduce dominance of the BSA in the calf serum. Table 3 below shows the estimated protein concentration in HPLC fractions of interest.

TABLE 3

| Fraction | Absorbance | Protein conc. after | Actual protein conc. |
|---|---|---|---|
| number | at 562 nm | dilution (μg/ml) | in fraction (μg/ml) |
| 4 | 0.13808 | 0.80 | 0.8 |
| 5 | 0.4262 | 8.17 | 40.85 |
| 6 | 0.50802 | 10.26 | 51.3 |
| 7 | 0.54022 | 11.09 | 110.9 |
| 8 | 0.81695 | 18.16 | 181.6 |
| 9 | 1.6895 | 40.48 | 404.8 |
| 10 | 0.64992 | 13.89 | 13.89 |
| 11 | 0.3678 | 6.68 | 6.68 |
| 12 | 0.65204 | 13.95 | 139.5 |
| 13 | 1.4152 | 33.47 | 33.47 |
| 14 | 0.30291 | 5.02 | 5.02 |

In preparation for obtaining SERS spectra from the fractions, 10 μl of each fraction along with 1× PBS and 1:10 diluted calf serum was spotted onto an aluminum substrate and left to air-dry for about two hours. No SERS signal was obtained from 1× PBS, although a strong SERS signal was obtained from 10% calf serum (fraction 9 containing 404.8 μg/mL calf serum). No SERS signal was obtained from other lower concentration calf serum fractions.

To obtain SERS signals, fractions of higher concentration were obtained by lyophilizing the low concentration fractions for about 5 hrs and re-solubilizing in water. Then SERS samples were obtained by spotting onto the aluminum substrate 10 μl of each fraction along with 2.5 mg/ml BSA (Roche), 3.33 mg/ml BAS (NEW). and 1:10 diluted calf serum. The spots were left to air-dry on the substrate for about 2 hrs. Table 4 below shows the concentration in the concentrated fractions.

TABLE 4

| | | Protein | | volume | | Vol of | |
|---|---|---|---|---|---|---|---|
| | Absorbance | concentration | Actual protein | of | Protein | water | final protein |
| Fraction | at | after dilution | concentration in | solution | content after | added | on substrate |
| number | 562 nm | (ug/ml) | fraction (ug/ml) | (ul) | lyophilization | (ul) | (ug) |
| 4 | 0.13808 | 0.80 | 0.8 | — | | | |
| 5 | 0.4262 | 8.17 | 40.85 | 380 | 15.5 | 10 | 15.5 |
| 6 | 0.50802 | 10.26 | 51.3 | 560 | 28.7 | 10 | 28.7 |
| 7 | 0.54022 | 11.09 | 110.9 | 620 | 68.8 | 20 | 34.4 |
| 8 | 0.81695 | 18.16 | 181.6 | 550 | 100 | 30 | 33.3 |
| 9 | 1.6895 | 40.48 | 404.8 | 1200 | 486 | 70 | 34.7 |
| 10 | 0.64992 | 13.89 | 13.89 | 1020 | 14.1 | 10 | 14.1 |
| 11 | 0.3678 | 6.68 | 6.68 | 985 | 6.5 | 10 | 6.5 |
| 12 | 0.65204 | 13.95 | 139.5 | 280 | 39 | 10 | 39 |
| 13 | 1.4152 | 33.47 | 33.47 | 580 | 19.4 | 10 | 19.4 |
| 14 | 0.30291 | 5.02 | 5.02 | 580 | 2.9 | 10 | 2.9 |

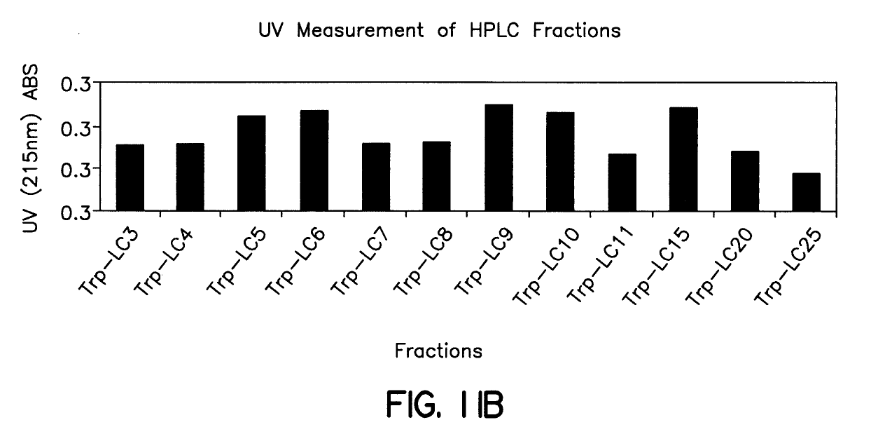

Raman signals were then obtained from the concentrated samples as shown in FIG. 10.

Based on these experiments, it was determined that highly concentrated protein sample should be used in HPLC separations to generate high concentration of proteins in fractions intended to be subjected to collection of SERS signals without the use of Raman tags or Raman labeling. The albumin depletion kit was also found to be ineffective for high concentration calf serum.

### EXAMPLE 4

It was hypothesized that peptides will give stronger SERS signals than proteins because access to SERS-active chemical structures is easier in smaller molecules. It was also hypothesized that peptides with different sequences may give unique SERS signatures that can be used for protein profiling. To test this hypothesis, the calf serum sample was first subjected to HPLC separation on a Zorbax GF-250 column (diol, size exclusion) using as the mobile phase 1× PBS, a sample volume of 20 μl, and an isocratic method, with run time of 20 min. The major albumin peak on the chromatograph (UV measurement) was confirmed as albumin by standard injection. The major absorption peaks of albumins are 205 nm and 225 nm. However a UV absorption profile of the main HPLC peak (BSA) of calf serum showed the absorption peaks are not uniform, indicating existence of multiple proteins in the peak.

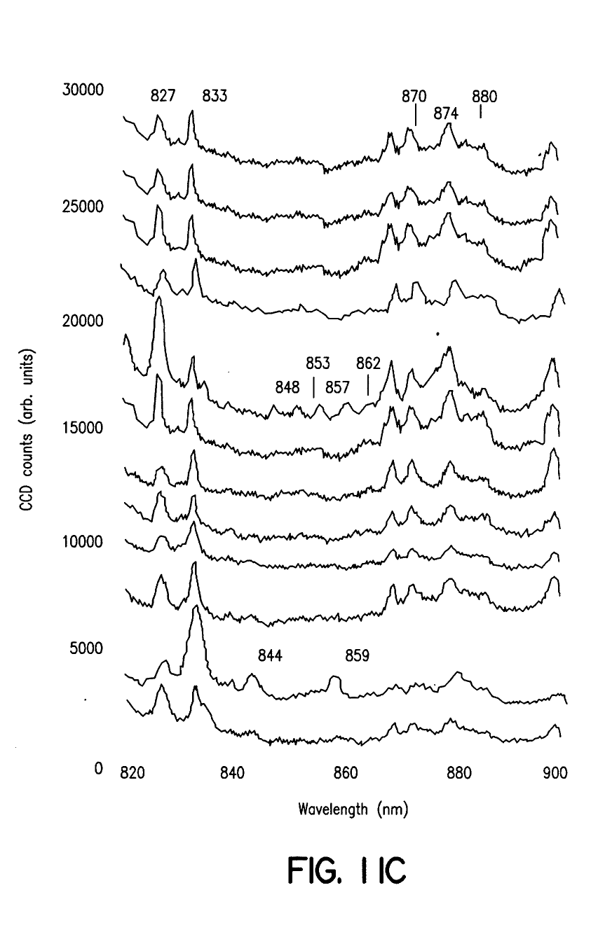

To determine the effect on SERS signals of working with peptides instead of whole proteins, the calf serum sample was subjected to trypsin digestion and the peptide mixture was separated with HPLC on a C18 column under acidic media. The aliphatic chain-coated silica in the C18 column binds peptides, and the TFA-CH₃CN mobile phase elutes the peptides according to their hydrophobicity. Acid in mobile phase is required to protonate the acid groups in the peptides, making them stay longer on the column. The HPLC separation used a sample volume of 50 μl and sample concentration of 1.4 μg/μl (≈21 μM) before digestion. Each fraction had ≈80 pmol of peptide. For HPLC a Zorbax SB-C18 column was used with a mobile phase buffer containing as Buffer A: 0. 1% TFA and as Buffer B: acetonitrile. The procedure for min. 0 to ≦5, 100% A; for min. 5 to ≦40, 100% A graded to 100% B. The chromatograph of HPLC separation of the trypsin-digested calf serum is shown in FIG. 11A. The HPLC peptide fractions were also measured for UV absorption (215 nm) (FIG. 11 B) and SERS spectra (FIG. 11C).

The results of these experiments show that proper protein sample preparation is important and protein fragmentation or denaturation is helpful in exposing Raman-active domains or amino acid residues to silver surfaces.

Although the invention has been described with reference to the above examples, it will be understood that modifications and variations are encompassed within the spirit and scope of the invention. Accordingly, the invention is limited only by the following claims.

1-35. (canceled)

36. A system for analyzing the protein composition of a complex mixture of proteins comprising:
a) a substrate having a plurality of discrete locations having a coating selected from a metal layer, a positively charged or negatively charged compound, and neutral or hydrophobic polymers for immobilization of proteins and protein fragments;
b) a sample containing at least one protein-containing compound;
c) a Raman spectrometer; and
d) a processor comprising an algorithm for analyzing the data generated by the spectrometer, said data comprising multiplexed signatures related to the protein composition of the sample.

37. The system of claim 36, wherein the Raman spectrometer is a scanner of SERS signals received consecutively from a plurality of the discrete locations.

38. A system for analyzing the protein composition of a complex mixture of proteins comprising:
a) a substrate comprising discrete locations, wherein one or more of said locations are associated with a capture probe/protein complex, said complex further comprising a Raman-active probe construct bound to the protein or the complex;
b) a Raman spectrometer arranged to interface with the substrate; and
c) a processor arranged to interface with the Raman spectrometer, said processor comprising an algorithm for analyzing Raman spectral signatures associated with capture probe/protein complexes comprising Raman-active probes.

39. The system of claim 38, wherein the capture probe is a primary antibody that binds specifically to the protein in the complex.

40. The system of claim 38, wherein the a Raman-active probe construct comprises a secondary antibody as probe and one or more Raman tags.

41. The system of claim 38, wherein the Raman-active probe construct is a COIN with a unique SERS signature and the Raman spectrum detected is a SERS spectrum.

42. The system of claim 38, wherein the substrate is coated with one or more organic or inorganic materials prior to immobilization of the proteins thereon.

43. The system of claim 38, wherein the protein is deposited at the discrete locations on the solid substrate by a procedure selected from contact writing, contact spotting, liquid spraying, and dry particle spraying.

44. The system of claim 38, wherein the substrate is aluminum.

45. The system of claim 38, wherein the discrete locations on the substrate comprise a material selected from gold, silver, copper, and aluminum metals, glass, silicon, and ceramic materials.

46. The system of claim 38, wherein the capture probe/protein complex comprising a Raman-active probe is further complexed with silver nanoparticles, in individual or aggregate forms.

47. The system of claim 46, wherein the Raman spectra are SERS spectra.

48. The system of claim 38, wherein the spectra contains information regarding a protein characteristic selected from a chemical bond, residue composition, residue structure, relative positions of residues, identity of the protein, and combinations thereof.

49. The system of claim 47, wherein the spectra contains information regarding a protein characteristic selected from a chemical bond, residue composition, residue structure, relative positions of residues, identity of the protein, and combinations thereof.
